(19) United States
(12) Patent Application Publication
(10) Pub. No.: US 2021/0353168 A1
(43) Pub. Date: Nov. 18, 2021

(54) Quantifying an Embedded PPG Signal to Noise Ratio Definition to Exploit Mediation of PPG Signal Quality on Wearable Devices

(71) Applicants: Laurence OLIVIER, Alpharetta, GA (US); LIFEQ GLOBALE LIMITED, Dublin (IE)

(72) Inventors: Laurence Olivier, Alpharetta, GA (US); Wikus Villet, Kuilsriver (ZA); Tim Verschaeve, Stellenbosch (ZA)

(21) Appl. No.: 17/280,983

(22) PCT Filed: Sep. 30, 2019
(86) PCT No.: PCT/US2019/053719

§ 371 Date: Mar. 29, 2021

Related U.S. Application Data
(60) Provisional application No. 62/738,878, filed on Sep. 28, 2018.

(51) Int. Cl. A61B 5/024; A61B 5/00; A61B 5/1455

(52) U.S. Cl. CPC A61B 5/02416; A61B 5/7203; A61B 5/7221; A61B 5/02438; A61B 2560/02; A61B 5/6802; A61B 5/6861; A61B 2562/162; A61B 5/1455

(57) ABSTRACT

Wearable data acquisition devices including sensors for detecting physiological signals are used to detect and measure various parameters pertaining to the health of the wearer. Through the use of algorithms, the data detected and measured by said sensors are processed to make inferences used to monitor the health of the wearer. An signal to noise ratio (SNR) calculation is used as a component of the sensitivity analysis of an algorithm. For example, a sleep algorithm will select a block of data, the SNR calculation will be done, with the average quality of the signal as result. The value associated with said signal quality may then be subjected to sensitivity analysis, which will yield a sleep accuracy value which, in turn, is indicative of the minimum signal quality which is required.

### FIELD

The disclosure relates to the quantification of signal quality of a photoplethysmograph (PPG) signal measured on any PPG—enabled wearable, nearable, ingestible and/or implantable sensors and/or so-called wearable devices, sensitivity analysis pertaining to said signal strength and, specifically, defining signal-to-noise (SNR)—ratio.

### BACKGROUND

In recent years, wearable devices, intended for human health related use, and their various associated sensors, algorithms and applications have become invaluable for the non-invasive and continuous detection, recording and subsequent digitizing of biological signals into data streams. The wearable device market has been experiencing such an immense growth streak that it is projected to grow from $20 billion in 2015, to $70 billion by 2025. Pre-eminent wrist-based devices fitted with sensors are now not only affordable to the general public, but are also omnipresent in the commercial sectors of fitness-, physical- and mental health monitoring, where the data collected by said device/s are proffered for use by medical facilities, health- and life insurers, and may even contribute greatly to the use and development of the Internet of Things.

Such devices may make use of many different types of sensors to detect and measure any said range of physiological signals, with varied accuracy. Types of sensors may include, for example, electrocardiograph (ECG), infrared (IR), PPG and pressure. Utilizing different types of sensors, as well as several other factors, contributes to the operational efficacy as well as the marketability of such devices. For example, the ability to determine the quality of the PPG signal under varying conditions of use (e.g. exercise/sleep/wake/active rest, etc.) may greatly improve functioning of said devices, as minimum signal quality may be guaranteed when this is achieved. However, to achieve this, a definition of signal quality, or Signal to Noise Ratio (SNR) must be outlined. Consequently, the optimal running of algorithms under any and all conditions may be guaranteed.

Currently, in excess of 266 companies worldwide produce wearables in the competitive market on a regular basis, with new contenders entering the market daily. These wearables are presented not only in the form of different models, but also as different versions of the same model, including improved versions of existing models—all in order to suit specific user needs as well as remaining on the cutting edge of the target market of the health- and fitness related sector of wearable technology. Competition is fierce amongst manufacturers to offer the product delivering not only the most comprehensive set of features, but also accurate inferences, prolonged battery life, best user experience, as well as ergonomic and aesthetically pleasing design. Therefore, when developing hardware and software to implement into said devices, being able to benchmark different devices against each other would mediate the tailoring of algorithms best suited to a specific device.

Furthermore, being able to guarantee adequate signal quality throughout use under any and all states of activity, for example sleep, wake, exercise, active rest, etc., presents challenges.

Duration of battery life is a principal consideration amongst manufacturers and consumers alike. As research techniques improve and user wants and needs increase, the number of built in features on these devices resultantly expand, which intensifies the need for additional battery power. Additionally, large, high resolution screens, variation in efficiency of computing algorithms and “always-on” features all contribute to the requirements of battery power.

Moreover, PPG technology is one of the foremost battery power consuming elements in modern wearables, as is on-device storing of raw data.

Whilst longer usage and shorter charging periods are equally important to users and manufacturers of wearables alike, the current rate of improvement in battery capacity is not keeping abreast with the rate of on-device components consuming said capacity.

Wireless charging has not advanced enough to be implemented successfully in conjunction with wearable devices worldwide.

### SUMMARY

Embodiments described herein are directed to an inherently stable, portable and scalable solution, addressing the challenges posed above, where signal quality may be determined under all conditions of use, adequate signal quality may be guaranteed, and battery power may be conserved on PPG enabled wearable devices without sacrificing either necessary or desired elements.

Wearable data acquisition devices include sensors for detecting physiological signals and are used to detect and measure various physiological parameters pertaining to the health of the wearer. However, in order to accurately and comprehensively monitor and make inferences as to the condition of the wearer's health, a definition of signal quality is required, which precedes the guaranteeing of adequate signal quality.

Methods are presented for quantifying PPG signal quality of any PPG—enabled wearable, nearable, ingestible and/or implantable sensors and/or so-called wearable devices, sensitivity analysis pertaining to said signal strength and, specifically, defining signal-to-noise ratio (SNR), to sufficiently provide signal quality for accurate algorithm performance and, subsequently, resulting in the conservation of battery power.

In some embodiments, a PPG frequency definition range defines a PPG signal component that resides in the signal frequency band ranging from 0.35 Hz to 7 Hz. In some embodiments, the noise frequency definition range defines the frequency components of the PPG above 7 Hz and is, therefore, classified as high frequency noise. In some embodiments, an SNR definition assesses a snapshot of data, utilizing between 3 and 4 seconds of data. In the embodiments, a PPG SNR determination method, discovered through extensive research, is performed to determine whether specific algorithms will perform optimally on any given device, in other words, the signal quality that is required to run specific algorithms is determined, also resulting in enhanced battery life.

Light reflected from the skin and/or the capillary blood vessels may either travel to a photodiode (Pd), or is processed by Complementary Metal-Oxide-Semiconductor (CMOS) technology. When working with a system that has its own controller, the SNR Controller (SNRC) may be used to feed such a controller, such that the system can be organized into modules.

The SNRC described herein enable the comparison of the PPG quality of two or more wearable devices, irrespective of the hardware configuration of said devices, and also enables a determination of which suitable algorithms may be compatible with a specified device.

The claimed invention includes a control system which uses a target- and calculated signal-to-noise ratio (SNR) value as input. The output is a target photo diode or analog-to-digital converter (ADC) current value which is fed to a closed-loop-controller (CLC), which drives the light-emitting diode (LED) intensity. The result is a system that locks in on a predefined signal quality while minimizing power consumption. More specifically, battery power is conserved to a significant extent during periods of sleep or other no or low motion activities, which, in turn, considerably extends the on-skin time for data collection.

A significant difference is also observed in the amount of light directly reflected by the skin of darker-skinned versus paler-skinned individuals. In a paler-skinned individual, a large amount of light is reflected, which is not modeled by PPG. In a darker-skinned individual, a large amount of light is absorbed, therefore, in a darker-skinned individual, much more light has to be emitted initially to achieve the same return than that of a paler-skinned individual. During sleep or other no or low motion activities, the PPG SNR definition is used to evaluate the signal quality of the varying component, adjusting the targets in order to equal the varying component between a paler- and a darker-skinned individual. Subsequently, by using this technique, a considerable amount of battery power may be conserved in the case of darker-skinned individuals during sleep or other no or low motion activities.

When an unknown device is plugged into a cloud-based computing platform, the associated data may be pushed through the SNR calculation algorithm, yielding a value associated with maximum signal quality achieved. From the available down-stream algorithms, specific ones with specified accuracy may be provided for a device which is to be potentially on-boarded, improving on current signal quality for said device. Therefore, a device which is to be potentially on-boarded may be quantified in this manner. During periods of sleep, where an individual remains stationary for an extended period of time and no movement effect is present, the PPG signal component may increase significantly. The claimed invention includes a control system which functions to conserve battery power to a significant extent during periods of sleep, while ensuring adequate signal quality. A significant difference is also observed in the amount of light directly reflected by the skin of certain darker-skinned versus paler-skinned individuals, which is not modeled by PPG. In paler-skinned individuals, a large amount of light is reflected, and not modeled, while in certain darker-skinned individuals, a large amount of light is absorbed. Usually, the device aims to match the magnitude of the non-varying reflection, therefore, in certain darker-skinned individuals, much more light has to be emitted initially to achieve the same return on non-varying reflection than in paler-skinned individuals, resulting in greater battery power consumption for certain darker-skinned users. During sleep, the controller evaluates the varying component, adjusting the targets in order to equal the varying component. Subsequently, by using this technique, a large amount of power may be conserved on darker-skinned individuals during sleep.

### BRIEF DESCRIPTION OF THE DRAWINGS/FIGURES

The accompanying drawings, which are incorporated herein and form a part of the specification, illustrate embodiments of the present disclosure and, together with the description, further serve to explain the principles of the disclosure and to enable a person skilled in the pertinent art to make and use the embodiments.

FIG. 1 illustrates a diagram of a high-level overview of the SNR system according to an embodiment of the present disclosure.

FIG. 2 illustrates an operational flow of a PPG SNR determination process in accordance with a PPG SNR frequency definition according to an embodiment of the present disclosure.

FIG. 3 depicts a diagram of a simplified SNR control scheme, as implemented in the CLC according to an embodiment of the present disclosure.

The present disclosure will be described with reference to the accompanying drawings. In the drawings, like reference numbers indicate identical or functionally similar modules.

### DETAILED DESCRIPTION

The following Detailed Description refers to accompanying drawings to illustrate exemplary embodiments consistent with the disclosure. References in the Detailed Description to “one exemplary embodiment,” “an exemplary embodiment,” “an example exemplary embodiment,” etc., indicate that the exemplary embodiment described may include a particular feature, structure, or characteristic, but every exemplary embodiment may not necessarily include the particular feature, structure, or characteristic. Moreover, such phrases are not necessarily referring to the same exemplary embodiment. Further, when a particular feature, structure, or characteristic is described in connection with an exemplary embodiment, it is within the knowledge of those skilled in the relevant art(s) to affect such feature, structure, or characteristic in connection with other exemplary embodiments whether or not explicitly described.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a computer of general purpose, as described below.

For purposes of this discussion, any reference to the term “module” shall be understood to include at least one of software, firmware, and hardware (such as one or more circuit, microchip, or device, or any combination thereof), and any combination thereof. In addition, it will be understood that each module may include one, or more than one, component within an actual device, and each component that forms a part of the described module may function either cooperatively or independently of any other component forming a part of the module. Conversely, multiple modules described herein may represent a single component within an actual device. Further, components within a module may be in a single device or distributed among multiple devices in a wired or wireless manner.

The following detailed description of the exemplary embodiments will so fully reveal the general nature of the disclosure that others can, by applying knowledge of those skilled in relevant art(s), readily modify and/or adapt for various applications such exemplary embodiments, without undue experimentation, without departing from the spirit and scope of the disclosure. Therefore, such adaptations and modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings herein.

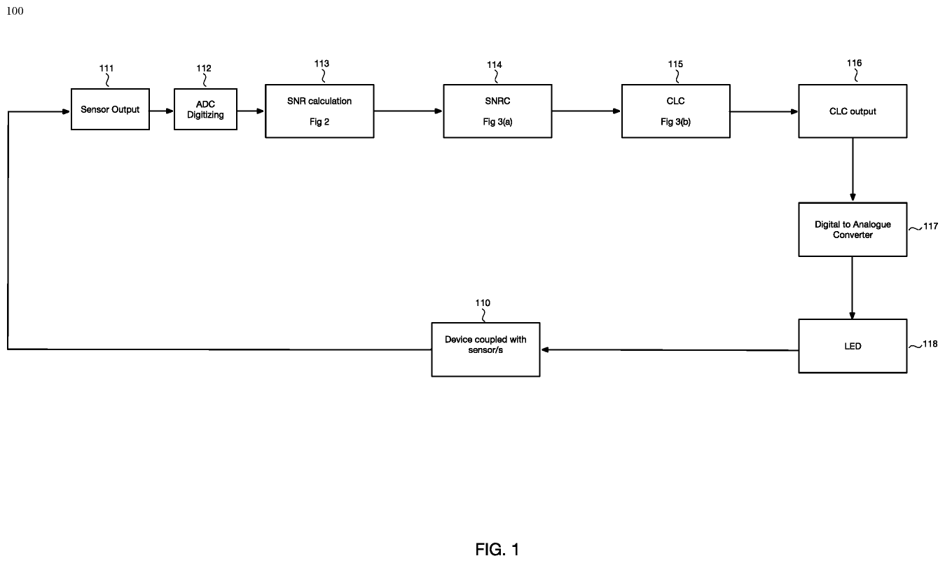

FIGS. 1 through 3 depict a method and a system for collecting physiological data on a wearable device and performing a SNR calculation on said data, on the wearable device. The system may include a wearable device that includes photoplethysmography (PPG) and/or other sensors used to measure various physiological signals, a mobile device such as a smart phone, tablet or laptop computer, wireless communications, and a cloud-based computing platform, to which third party servers such as insurers or medical group entities may have access.

FIG. 1 is a flowchart showing exemplary operational steps of an SNR system operational control 100. With reference to FIG. 1, at operation 110 a wearable, nearable, ingestible and/or implantable device coupled with a sensor or sensors acquires one or more physiological signals. At operation 111, the one or more acquired physiological signals is output in analog form. At operation 112, the output is digitized by means of an Analog to Digital Converter (ADC), which allows the digital signal to be used in a microcontroller, integrated circuit and/or other computing device.

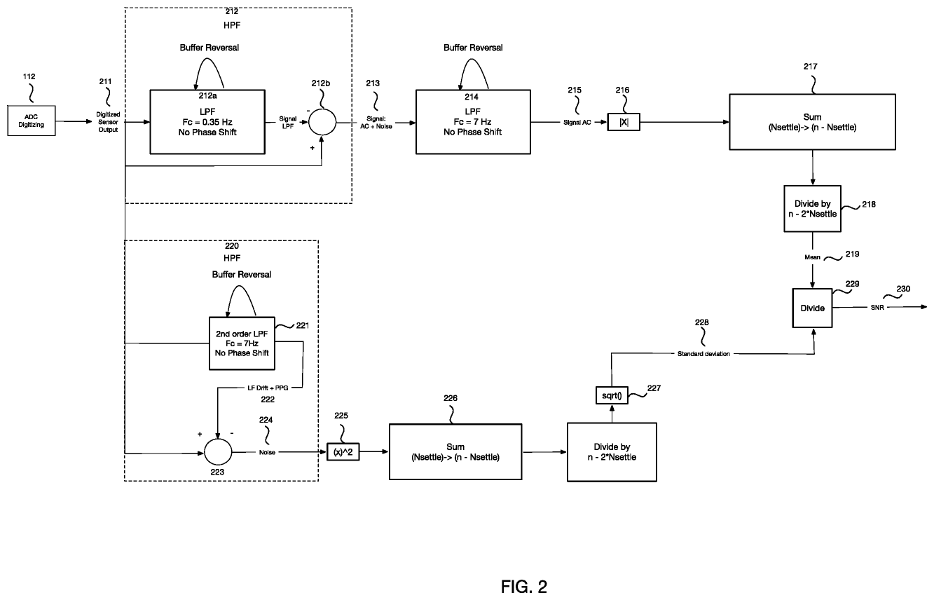

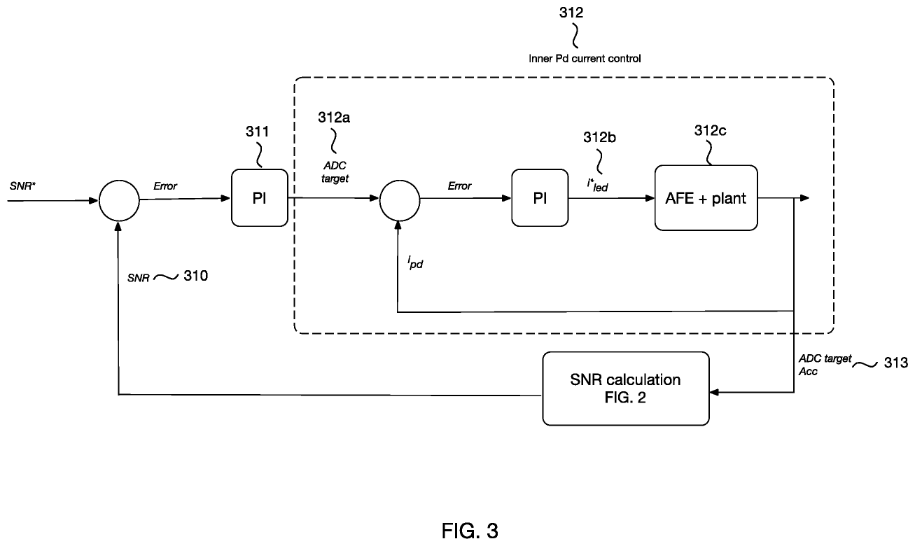

At operation 113, the digitized signal is used to calculate the PPG Signal to Noise Ratio (SNR) by a PPG SNR determination method, described in detail below with reference to FIG. 2. At operation 114, the PPG SNR is output into a SNR Controller (SNRC) with reference to FIG. 3(a). At operation 115, the SNRC controls the SNR based on the PPG SNR determination method and a target value is output to the Closed Loop Controller (CLC), as described in detail below with reference to FIG. 3(b). At operation 116, the CLC generates a digital output. At operation 117, the digital output is fed into a Digital to Analog Converter (DAC). At operation 118, the DAC drives Light Emitting Diodes (LEDs) in accordance with the digital output generated by the CLC.

That is, the CLC determines the brightness at which the LEDs should be driven to complete the entire loop of controlling the SNR to PPG, whereby the LED illumination brightness is fed to one or more PPG-enabled wearable, nearable, ingestible and/or implantable sensors and/or so-called wearable devices coupled with a sensor or sensors 110, followed by sensor output 111 entering the system as previously described.

FIG. 2 is a method for calculating PPG SNR 230. At 211, a physiological signal is detected at 110 (See FIG. 1). The signal can be reflected by the skin, or any other relevant tissue layer of the user, where after the sensor output (e.g., as described above with reference to operation 111 of FIG. 1) is measured and stored in the memory of a microcontroller unit (MCU), FPGA or other programmable logic device. The signal is digitized (e.g., as described above with reference to operation 112 of FIG. 1) and output as digitized sensor output 211. The signal is filtered by means of a high pass filter 212, which then leaves the PPG AC component plus high frequency noise 213. The high pass filter is implemented by means of a low pass filter (LPF) 212a, and a signal subtraction 212b. The low pass filter 212a has a cutoff frequency according to a first PPG frequency definition. In some embodiments, a first PPG frequency definition may be, for example, 0.35 Hz. Because this is a digital filter, a buffer reversal is done to eliminate phase shift. The PPG AC component plus high frequency noise 213 is passed through a second LPF 214 having a cutoff frequency according to a second PPG frequency definition. In some embodiments, a second PPG frequency definition may be, for example, 7 Hz. Another buffer reversal is implemented to eliminate phase shift and the resulting signal at 215 corresponds to the AC component only. That is, the signal AC 215 includes the PPG signal without noise, after the low pass filter removes said high frequency noise component. This is followed by a sequence necessary in managing digital data where the PPG signal 216 is determined based on an absolute value of signal AC 215. The resultant signal is summed 217 over the entire buffer minus the filter settling time, divided 218 by the number of values summed, to provide the mean signal AC 219 for this buffer. Thereby, the mean signal PPG 219 is determined.

The next step is to determine the noise component in accordance with the noise frequency definition range, as shown in FIG. 2. The original digitized signal 211 enters a second HPF 220 where it is passed through a second order LPF 221 with a cutoff frequency of 7 Hz. As previously discussed, a buffer reversal is implemented to eliminate phase shift. LPF 221 removes the high frequency (HF) noise from the component, resulting in the PPG component value and a low frequency (LF) drift on the PPG 222. The LF drift and PPG component value (see 222) is subtracted 223 from the original signal 211 to determine noise 224. Each noise value 224 is squared 225, summed 226 and divided 227 by the number of samples, to acquire a mean error value, similar to the algorithm above (e.g., 217 and 218).

The mean 219 of the signal and the standard deviation 228 are thereby determined. An SNR calculation is then performed, by which the mean 219 is divided 229 by the standard deviation 228 to determine the SNR of the PPG to the system noise. Thus, SNR 230 is determined within the range of said frequencies defined between a first PPG frequency definition and a second PPG frequency definition. In exemplary aspect, the first and second PPG frequency definitions range from 0.35 Hz up to 7 Hz. However, in other aspects, the range of the first and second PPG can vary. Referring to the exemplary aspect discussed above, frequencies outside of the first PPG frequency definition and the second PPG frequency definition, e.g., those below 0.35 Hz or above 7 Hz, are thus classified as noise. Accordingly, the characteristics of the filters 212 and 220 contribute significantly to determining the cutoff frequencies. The filters are implemented, using only second and third order filters, on an embedded level to minimize processing power consumption. Furthermore, the filters are manually constructed.

FIG. 3 depicts a schematic representation of a simplified SNR control scheme as implemented in the inner control loop (ICL) 312, which may be combined with the above embodiments of FIGS. 1 and 2. For example, ICL 312 may be an embodiment of at least part of a CLC described above (e.g., with respect to operation 115 of FIG. 1). The control loop may be driven by a photodiode or ADC target, for example, depending on the PPG sensing technology. The target value obtained at operation 112 can be used as input for ADC target 312a to determine an LED illumination brightness as output 312b.

The SNR calculation can be used as a component of the sensitivity analysis of an algorithm. For example, a sleep algorithm will select a block of data and the SNR calculation will be performed, with the average quality of the signal as the result. The value associated with said signal quality may then be subjected to sensitivity analysis, which will yield a sleep accuracy value which, in turn, is indicative of the minimum signal quality which is required. In some embodiments, when an unknown device is plugged into a cloud-based computing platform, the associated data may be pushed through the proposed SNR calculation algorithm, yielding a value associated with maximum signal quality achieved. From the available down-stream algorithms, certain algorithms with specified accuracy may be provided for a device which is to be potentially integrated, improving on current signal quality for said device. Therefore, a device which is to be potentially integrated to the platform may be quantified in this manner.

When an individual remains stationary for an extended period of time and no movement effect is present, for example when sleeping, the PPG signal component, as defined by the SNR definition, may increase significantly, which means that the amount of LED current that is being pushed through is at the same conditions as that of an individual training, for example, on a treadmill. Therefore, by pre-determining the required conditions for, say, an algorithm used during periods of sleep, and adjusting accordingly, the amount of current that is applied may be adjusted by the controller to ensure the required minimum is maintained. A significant difference is also observed in the amount of light directly reflected by the skin of certain darker-skinned versus paler-skinned individuals, which is not modeled by PPG. In paler-skinned individuals, a large amount of light is reflected, and not modeled, in contrast with darker-skinned individuals. Usually, the device aims to match the magnitude of the non-varying reflection, therefore, in darker-skinned individuals, much more light has to be emitted initially to achieve the same return on non-varying reflection than in paler-skinned individuals, resulting in greater battery power consumption for users with darker skin. During sleep, the controller evaluates the varying component, adjusting the targets in order to equal the varying component. Subsequently, by using this technique, a large amount of power may be conserved on darker-skinned individuals during sleep. This is achieved through the controller decreasing or increasing the number of photons generated on the photo diode in keeping with the known required signal quality, where after the values calculated for the signal magnitude and the noise component are used to quantify the quality of the measured PPG signal. This correlates with the amount of LED power that is used and, accordingly, the LED power is decreased or increased to a level where the signal quality produced is at the required minimum.

Derived values, as per photo diode (Pd) current and/or raw sensor output, may serve as input for the calculation. In an aspect, the SNR controller (SNRC) is activated after 6 successive SNR calculations in the absence of excessive accelerometer movement, with the SNR calculation being performed on an embedded level. The measured DC signal from the sensor is varied through the use of a proportional integral (PI) controller, and should accelerometer movement higher than a predefined threshold be detected, the controller is shifted out of SNRC mode and back to standard DC tracking. The SNRC uses the SNR calculations to force the signal quality (either up or down) to a predefined target number, while keeping the signal quality within safe operating bounds. This pertains to the number of LED photons generated which, in turn, generates a certain number of photons on the Pd or CMOS. The DC of the measured signal is controlled by the controller in question. In an aspect, the controller may be set to adjust the current to 6 μA, where after the controller will set the LEDs to track and supply the desired current. At the back end, the signal can be amplified. A second controller loop is then employed to control the amount of gain used to amplify the amount of current into a large PPG signal. Both these controller loops constitute the CLC.

Components in the measured reflection include:
a. Direct Current (DC) Offset, which does not contribute to the SNR calculation and, therefore, is suppressed
b. Low frequency drift, which includes breathing rate and other artefacts, and is suppressed in the SNR calculation
c. Signal variation generated by HR, which is used for the SNR calculation and is isolated from the rest of the signal with as little suppression as possible
d. High frequency noise, which constitutes component and measurement noise

The SNR calculation may be explained as follows:

### Signal:

$$μ = \frac{Σ \vert AC \vert}{n} ;$$

where n equals the number of samples in the buffer

### Standard Noise Deviation:

$$σ = \sqrt{\frac{{Σ ( noise )}^{2}}{n}} ;$$

where n equals the number of samples in the buffer

$$S N R = \frac{μ}{σ}$$

$$S N {R}_{d b} = 10 {log}_{10} ( \frac{μ}{σ} )$$

The equation applied for calculating the signal quality on-device may also be used to determine the percentage fault at a certain signal quality. Consequently, by varying the amount of power that is used, the controller may be programmed to ensure that the pre-specified signal quality is delivered throughout for a specified algorithm where the required accuracy is known.

Storing all raw data on-device exhausts battery power. By employing the SNR calculation algorithm, only data which registers at a specified accuracy target level is stored. In this manner, a quality metric is calculated on-device without the requirement for storing said raw data.

Certain 1 Hz downstream metrics, for example heart rate and pulse wave form, are calculated from 25 Hz PPG data. However, due to storage constraints, large amounts of 25 Hz data are discarded. As a result, analyzing the quality of the PPG data with which the embedded 1 Hz data is computed, is impossible. The embodiments disclose herein provide a solution in terms of the embedded SNRC being logged periodically with said 1 Hz data and thus determining when the signal quality of the PPG data is adequate to be logged.

### Improvement on Wear Detection

A combination of the CLC and SNRCs improves wear detection in the following manner: A wear detection trigger is fed to the CLC which, in response, increases the target Pd current significantly and, therefore, also increases the illumination light. As the calculated SNR values are fed through to the CLC, the CLC evaluates the signal quality and performs an educated decision on when to decrease the target Pd to its normal value. This contributes to rejection of wear detection triggers that are the result of poor signal quality, rather than off-skin events.

The exemplary embodiments described herein are provided for illustrative purposes and are not limiting. Other exemplary embodiments are possible, and modifications may be made to the exemplary embodiments within the spirit and scope of the disclosure. Therefore, the Detailed Description is not meant to limit the disclosure. Rather, the scope of the disclosure is defined only in accordance with the following claims and their equivalents.

What is claimed is:

1. A method for processing at least one physiological signal, the method comprising
(a) determining a photoplethysmograph (PPG) component from the at least one physiological signal;
(b) determining a PPG noise component from the at least one physiological signal; and
(c) calculating a PPG signal-to-noise ratio (SNR) in order to determine signal quality.

2. The method of claim 1, wherein the at least one physiological signal is collected using a device comprising at least one sensor.

3. The method of claim 2, wherein the at least one sensor comprises a PPG sensor.

4. The method of claim 2, wherein the device comprises a wearable device, a nearable device, an ingestible device or an implantable device.

5. The method of claim 2, further comprising performing sensitivity analysis of an algorithm for the device using a PPG controller.

6. A system for calculating a photoplethysmograph (PPG) signal-to-noise ratio (SNR), the system comprising:
at least one computing device, comprising:
a memory; and
a processor coupled to the memory,
wherein the processor, based on instructions stored in the memory, is configured to perform a determination of the PPG SNR of at least one PPG enabled physiological measurement captured using at least one sensor coupled with the at least one computing device.

7. The system of claim 6, wherein the determination of the PPG SNR is implemented with a PPG controller.

8. The system of claim 7, further comprising performing sensitivity analysis of an algorithm for the at least one computing device using the PPG controller.

9. The system of claim 6, wherein the at least one computing device comprises a mobile device, smart phone, tablet, laptop computer, wireless communications device or a cloud-based computing platform.

10. The system of claim 6, wherein the at least one sensor is coupled with a wearable, nearable, ingestible or implantable device.

11. The system of claim 6, wherein the at least one sensor comprises a photoplethysmography sensor.

12. The system of claim 6, wherein the at least one sensor comprises a nearable, ingestible or implantable sensor.

13. A system for processing at least one physiological signal, the system comprising:
(a) a device comprising at least one light emitting diode driven by a closed-loop controller;
(b) at least one sensor coupled to the device, wherein the at least one physiological signal is acquired via the at least one sensor and output in a first analog form;
(c) an analog-to-digital converter coupled to the device, wherein the first analog form is digitized and used in a signal-to-noise ratio calculation and wherein the output of the signal-to-noise ratio calculation is inputted into a signal-to-noise ratio controller and the closed-loop controller; and
(d) a digital-to-analog converter coupled to the device, wherein the output of the closed-loop controller is converted to a second analog form and used to determine light output of the at least one light emitting diode.

14. The system of claim 13, wherein the device comprises a wearable device, a nearable device, an ingestible device or an implantable device.

15. The system of claim 13, wherein the signal-to-noise ratio calculation comprises removing noise components outside of a specified range from the at least one physiological signal.
